(19) United States
(12) Patent Application Publication
(10) Pub. No.: US 2024/0393318 A1
(43) Pub. Date: Nov. 28, 2024

(54) METHOD FOR STUDYING THE BEHAVIOUR OF A CELL SAMPLE

(71) Applicants: ECOLE POLYTECHNIQUE, Palaiseau (FR); CENTRE NATIONAL DE LA RECHERCHE SCIENTIFIQUE, Paris (FR); INSTITUT NATIONAL DE LA SANTE ET DE LA RECHERCHE MEDICALE, Paris (FR); UNIVERSITE PARIS EST CRETEIL VAL DE MARNE, Creteil (FR)

(72) Inventors: Jongwook KIM, Paris (FR); Thierry GACOIN, Bures sur Yvette (FR); Marcel FILOCHE, Bourg la Reine (FR); Bruno LOUIS, Creteil (FR); Emilie BEQUIGNON, Charenton le Pont (FR)

(21) Appl. No.: 18/694,185

(22) PCT Filed: Sep. 19, 2022
(86) PCT No.: PCT/EP2022/075972

§ 371 Date: Mar. 21, 2024

(30) Foreign Application Priority Data
Sep. 23, 2021 (FR) FR2110013

(51) Int. Cl. G01N 33/50; C12M 1/34; C12M 3/06; G01N 21/64

(52) U.S. Cl. CPC G01N 33/5005; C12M 23/16; C12M 41/46; G01N 21/6428; G01N 21/6458; G01N 2021/6439

(57) ABSTRACT

A method for studying the behaviour of a cell sample contained in a medium containing a plurality of anisotropically shaped nanoparticles dispersed therein, the method including: (i) determining at least one nanoparticle orientation characteristic in a measurement zone at the interface between the fluid medium and the cell sample, the orientation resulting at least partially from the interaction of the fluid medium and the biological material; (ii) determining a mean shear rate characteristic of the fluid medium in the measurement zone from said at least one nanoparticle orientation characteristic determined in this measurement zone; and (iii) determining a characteristic of the cell sample from the mean shear rate thus determined in the measurement zone.

The present invention relates to a process for studying the behavior of a cell sample contained in a fluid medium. The invention also relates to the corresponding study device.

### TECHNICAL FIELD

Currently, studying the behavior of a cell sample, notably its viability and/or functionalities, usually requires the use of high-resolution optical or electron microscopy tools, combined with complex image analysis and/or genetic analysis. Such procedures are slow and costly, making them unsuitable for clinical studies, notably in the case of in vitro evaluation of biological cells collected in routine healthcare practice.

By way of example, mucociliary clearance is one of the main means of defense via which the lungs continuously evacuate inhaled particles from the airways. Unwanted particles are trapped by the mucus covering the airway epithelium. The airborne contaminants and mucus are then carried along the airways by the synchronous beating of cilia present on the airway epithelium, until they are expelled into the oesophagus. The absence or impairment of this clearance may lead to chronic obstructive mucus accumulation, conditions seen not only in rare congenital diseases such as cystic fibrosis or primary ciliary dyskinesia (PCD), but also in much more common diseases such as chronic rhinitis, asthma and chronic obstructive pulmonary disease (COPD). This absence or impairment of mucociliary clearance may be due to dysfunction of individual ciliary beating (movement or motricity) and/or ciliary coordination.

in vivo observation of ciliary beating so as to evaluate mucociliary clearance is difficult. In the past, in vivo evaluation of mucociliary clearance was performed via techniques using saccharin, a drop of blue marker or radioactive tracer clearance. Microoptical coherence tomography has also been proposed. However, due to the constraints imposed by these various techniques, notably patient cooperation for the saccharin test, the invasiveness for endoscopic examination and/or inhalation of radiopharmaceutical elements, they have been all but abandoned in current clinical practice. Currently, the most commonly used method for evaluating ciliary beating is ex vivo study by bright-field microscopy, often combined with high-speed video-microscopy (HSVM) analysis of the ciliary beating frequency of ciliated cells, obtained by nasal or bronchial brushing. Another method used for evaluating mucociliary clearance consists in tracking the movement of fluid displacement marker microbeads generated by ciliary beating using particle imaging velocimetry (PIV). This method, described in the article Bottier, M. et al. “A new index for characterizing micro-bead motion in a flow induced by ciliary beating: part II, modeling”, PLOS Comput. Biol. 13 (7): e1005552 (2017). This method is based on a theoretical model of the flow movement induced by the active beating of the cilia, making it possible to evaluate the shear stress exerted by the cilia on the medium. This shear stress proves to be a reliable index for characterizing the efficiency of ciliary beating. However, this experimental method of evaluating shear stress has low spatial resolution, and has proven to be time-consuming and difficult to implement in a routine clinical context.

Recent studies described in the article Kim, J., Michelin, S., Hilbers, M. et al. “Monitoring the orientation of rare-earth-doped nanorods for flow shear tomography”, Nature Nanotech 12, 914-919 (2017) allow mapping of the shear rate in a microchannel in which a fluid is flowing, via the use of suspended europium-doped lanthanum phosphate (LaPO₄:Eu) nanorods. This method has never been used to evaluate the behavior of cell samples in a fluid, and its implementation requires the resolution of numerous problems, inter alia the stability of the nanorods in the medium suitable for cell samples, the cellular non-toxicity of the nanorods, and the production of a spatiotemporal resolution allowing the study of the dynamic behavior of the cell sample at the microscopic level.

There is thus a need for a process for studying the behavior of a cell sample in a fluid, which is fast, has good resolution, and is both easy to implement in clinical practice and inexpensive.

### DISCLOSURE OF THE INVENTION

The invention meets this need via a process for studying the behavior of a cell sample contained in a fluid medium containing a plurality of anisotropically-shaped nanoparticles dispersed therein, the process involving:
(i) determining at least one orientation characteristic of the nanoparticles in a measurement zone at the interface between the fluid medium and the cell sample, the orientation resulting at least partly from the interaction of the fluid medium and the cell sample,
(ii) determining a shear characteristic of the fluid medium in the measurement zone from the nanoparticle orientation characteristic determined in this measurement zone,
(iii) determining a characteristic of the cell sample from the shear characteristic thus determined in the measurement zone.

The term “shear characteristic” means the measurement of a characteristic of the fluid velocity gradient in the fluid medium at the interface with the cell sample, notably its direction and/or value. Such a measurement allows the shear force exerted by the cell sample on the fluid medium to be characterized.

The term “anisotropically shaped nanoparticles” refers to particles with an aspect ratio defined as the ratio of the length to the largest transverse dimension greater than 1, i.e. substantially elongated in at least one direction.

The term “dispersed” means that the nanoparticles are suspended in the medium without aggregating together.

The term “nanoparticle orientation” means that the nanoparticles, by virtue of their anisotropic shape, can orient themselves statistically in the fluid medium as a function of the shear stresses to which they are subjected. The higher the shear stress they undergo, the more the nanoparticles will orient themselves in a similar direction corresponding substantially to the direction of fluid flow, and the narrower the orientation distribution will be around this direction.

As explained above, the nanoparticles orient themselves in the fluid medium according to the shear force they experience. Determining at least one nanoparticle orientation characteristic in a measurement zone allows the shear rate in the measurement zone to be determined and information to be deduced regarding a cell sample characteristic, notably the overall ciliary beating efficiency, for example by comparison with a predetermined local shear rate for a healthy cell sample.

Preferably, the process is an ex vivo or extemporaneous process.

The process is non-therapeutic per se.

### Cell Sample

Preferably, the cell sample is composed of at least one cell cluster notably chosen from beating ciliated epithelial cells, in particular a sample of a border of a ciliated epithelium, flagellated cells, embryonic cells, blood cells, reproductive cells, red blood cells and immune system cells.

The cell sample may be of human, animal or plant origin.

### Fluid Medium

Preferably, the fluid medium is liquid, notably aqueous.

Preferably, the fluid medium includes a physiologically acceptable solution for the cell sample, notably Dulbecco's modified Eagle's medium (DMEM), to maintain the viability of the cell sample. Where appropriate, the fluid medium may contain nutrients for the cell sample and constitute a culture medium.

Preferably, fluid circulation is induced by movement at the surface of the biological sample without external action. Such circulation may be, for example, the result of cilia movements on the surface of the cell sample.

### Nanoparticles

Preferably, the nanoparticles are nanorods, i.e. nanoparticles of generally elongated shape extending mainly along a curvilinear or, preferably, rectilinear directrix. In this case, the aspect ratio is the ratio of the length, measured along this directrix, to the width, the width being the largest dimension that can be measured in all transverse planes (perpendicular to the directrix) along the directrix.

Preferably, the aspect ratio of the nanoparticles is greater than or equal to 3, better still greater than or equal to 10.

Preferably, the nanoparticles are configured to emit photoluminescent radiation and/or are birefringent.

Preferably, the nanoparticles exhibit polarized photoluminescence emission.

Preferably, the nanoparticles are rare-earth-doped nanorods. The nanoparticles may be oxide or fluoride, notably lanthanum phosphate (LaPO₄), sodium yttrium fluoride (NaYF₄) or derivatives thereof, doped with rare-earth metals, notably europium. The nanorods may have crystalline symmetry, notably symmetry along an axis parallel to the nanorod directrix. The nanorods are preferably europium-doped LaPO₄ and may be in the rhabdophane crystal phase, or preferably in the monazite crystal phase.

Such nanorods each emit photoluminescent radiation which is strongly polarized as a function of the orientation of the nanorods. It is thus possible, as described in the article Kim, J., Michelin, S., Hilbers, M. et al. “Monitoring the orientation of rare-earth-doped nanorods for flow shear tomography”, Nature Nanotech 12, 914-919 (2017), by measuring the polarized fluorescence radiation at different polarization angles of the nanorods in a measurement zone containing several nanorods, notably several hundred nanorods, to determine at least one orientation characteristic of the nanorods. A difference in the shape of the spectrum at different polarization angles is characteristic of a particular orientation of the nanoparticles and thus of the presence of a shear force in the measurement zone in the fluid medium.

Such rods are also birefringent, and measurement of at least one characteristic of the birefringence profile in a measurement zone may also allow determination of an orientation characteristic of the nanorods and deduction of a shear characteristic in the measurement zone.

Preferably, the nanoparticles have an average length of less than or equal to 1 μm, better still less than or equal to 200 nm. Preferably, the standard deviation of the nanoparticle length distribution is less than or equal to 100 nm. Such a nanoparticle distribution allows good spatiotemporal measurement resolution and good sensitivity of nanoparticle orientation to shear in the fluid medium.

Preferably, the nanoparticles include dispersion-stabilizing linking molecules on the surface, notably linking molecules with amine, silane (PEG-silane) or alendronic acid end groups. Such molecules improve the stability of the nanoparticle dispersion in the biological fluid medium by isolating the nanoparticle core from the fluid medium. Furthermore, they have low toxicity for the cell sample.

Preferably, the nanoparticle concentration in the fluid medium is less than or equal to 10% as a volumetric fraction of the fluid medium.

### Photoluminescence

Preferably, the nanoparticles are photoluminescent and the step for determining the orientation characteristic of the nanoparticles involves:
photoluminescence excitation of the nanoparticles by a light source, causing the nanoparticles to emit photoluminescent light, and
measuring at least two items of spectral information of the polarized photoluminescence light at one or more different polarizations in the measurement zone,
determining the orientation characteristic of the nanoparticles from the measurements of the at least two items of spectral information of the polarized photoluminescence light at one or more different polarizations.

Preferably, measurement of the two items of spectral information involves optical detection of photoluminescence light from the nanoparticles using an optical microscopy system, notably a confocal microscopy system. The microscopy system may include a confocal optical microscope, and one or more optical sensors of the light emitted by photoluminescence from the nanoparticles. The optical sensor(s) may be photodiodes, notably photomultiplier tubes (PMTs), avalanche photodiodes (APDs), combined with spectral optical filters or wavelength-resolved spectrometers.

The process may involve rotating a polarizing filter to change the polarization angle, notably arranged between the fluid medium and the optical system, notably the optical sensor(s) to allow measurements of at least two items of spectral information of the polarized photoluminescence light at one or more different polarizations.

Preferably, the excitation light source is a laser source configured to excite the nanoparticles at a predetermined optimum wavelength corresponding notably to an excitation peak, notably between 250 nm and 550 nm, notably for LaPO₄:Eu at a wavelength of 394 nm.

As a variant, the excitation light source emits light with a broad wavelength spectrum, notably white light.

In the case of LaPO₄:Eu, the at least two items of spectral information measured may correspond to the spectra between 570 and 720 nm in which the transition bands of the Eu³⁺ ion are located.

The process may involve measuring the intensity of light polarized at one or more different polarization angles at least two different given wavelengths, the given wavelengths corresponding in the spectrum of light emitted by the nanoparticles to two different intensity peaks of the photoluminescence light spectrum, notably for LaPO₄:Eu nanorods, being
between 580 nm and 590 nm, and between 590 nm and 600 nm, respectively,
between 610 nm and 617 nm and between 617 and 630 nm, respectively, or
between 680 nm and 690 nm and between 690 and 710 nm, respectively.

Preferably, the angle between the two polarization angles is greater than or equal to 10°, better still greater than or equal to 30°, even better still greater than or equal to 60°, for example equal to 90°.

The process may involve identifying a difference in intensity of the polarized light at the two different polarization angles at a given wavelength corresponding to a preferential orientation of the nanoparticles in a particular direction.

The process may involve measuring a plurality of spectral information of the polarized photoluminescence light, notably the intensity of the polarized light at a given wavelength range or the spectral profile of the polarized light, at several different polarization angles.

As a variant, the process involves measuring the birefringence profile of the nanoparticle distribution in a measurement zone, determining a nanoparticle orientation characteristic from the birefringence measurement of the nanoparticle distribution in a measurement zone.

Preferably, the measurement of the spectral information of the photoluminescence light polarized at one or more different polarizations takes place temporally after the photoluminescence excitation of the nanoparticles by a light source, notably at least 5 microseconds, better still at least 10 microseconds, afterwards to eliminate parasitic fluorescence.

### Orientation

Preferably, the shear characteristic determined in the measurement zone is the mean shear value in the measurement zone.

Preferably, the process involves determining at least two characteristics of the nanoparticle orientation, the nanoparticle orientation and the associated order parameter characteristic of the dispersion of the nanoparticle orientation relative to the orientation in the measurement zone. To this end, the determination method is as described in the article Kim, J., Michelin, S., Hilbers, M. et al. “Monitoring the orientation of rare-earth-doped nanorods for flow shear tomography”, Nature Nanotech. 12, 914-919 (2017), incorporated herein by reference.

Preferably, the process involves determining the shear direction and shear value in the measurement zone from the two nanoparticle orientation characteristics determined in this measurement zone.

### Measurement Zone

Preferably, the measurement zone corresponds to the focal volume of the optical microscopy system. It may have a volume of less than or equal to 3 μm³, preferably 1 μm³.

### Dynamic Mode

The process may involve determining the dynamic shear characteristic of the fluid medium in the measurement zone via the method described previously from continuous measurements over time in the same measurement zone, and determining a characteristic of the cell sample from the dynamic shear characteristic thus determined in the measurement zone.

### Scan Mode

The process may involve:
scanning the fluid medium in at least two directions by the measuring system, determining at least one nanoparticle orientation characteristic at each position of the measuring system,
mapping the shear characteristic in the fluid medium from at least one nanoparticle orientation characteristic measured at each position of the measuring system.

### Characterizing the Cell Sample

Preferably, the process involves comparing the shear characteristic in the determined measurement zone or the mapping of the shear characteristic to a reference shear characteristic or a reference mapping of the pre-established shear characteristic, notably for a reference cell sample.

The process may involve characterization, notably the efficiency of the cell sample from the shear characteristic in the determined measurement zone or from the mapping of the determined shear characteristic.

Preferably, the cell sample is of ciliated epithelial cells and the process involves determining the ciliary beating efficiency of said cells by comparison of the determined shear rate or the determined shear rate mapping and a pre-established reference shear rate or mapping corresponding to normal ciliary beating of the ciliated epithelial cells.

Preferably, the cell sample is obtained by nasal brushing.

Preferably, the time between production of the cell sample and measurement is less than or equal to 3 hours.

### Device

A subject of the invention is also a device for studying a cell sample, notably for performing the process defined above, including:
a fluidic chamber including:
   a fluid medium, and
   a plurality of anisotropically shaped nanoparticles dispersed in the medium,
a system for measuring at least one item of information characteristic of the orientation of the nanoparticles in a measurement zone at the interface between the fluid medium and the cell sample.

The device may include or be connected to a processor configured to determine at least one characteristic of nanoparticle orientation in the measurement zone.

Preferably, the chamber is a microfluidic chamber.

The device may include the cell sample in the fluid medium.

The properties of the process described above apply to the device, alone or in combination.

Preferably, the device includes or is connected to a processor configured to determine the shear characteristic in the measurement zone from information characteristic of the nanoparticle orientation determined by the measurement system in this measurement zone, notably from the determined nanoparticle orientation characteristic.

Preferably, the measurement system includes a system for scanning in at least two directions, better still three directions, to move the measurement system between several measurements so as to take measurements in several different measurement zones. The processor may determine mapping of the shear rate in the fluid medium as a function of the orientation information so as to map the shear rate in the fluid medium from the orientation information of the nanoparticles measured by the measurement system in different measurement zones.

Preferably, the measurement system includes:
a light source for photoluminescent excitation of the nanoparticles,
an optical microscopy system, notably including:
   a confocal optical microscope, and
   one or more optical sensors for the light photoluminescently emitted by the nanoparticles,
a rotating mobile polarizing filter positioned between the chamber and the optical microscopy system, notably the optical sensor(s).

The optical sensor(s) may be configured to take a delayed measurement relative to the photoluminescent excitation light source.

### BRIEF DESCRIPTION OF THE DRAWINGS

FIG. 1 schematically represents an example of a microfluidic chamber,

FIG. 2 schematically represents an example of a device, and

FIG. 3 schematically represents the ciliary beating of ciliated epithelial cells in three dimensions.

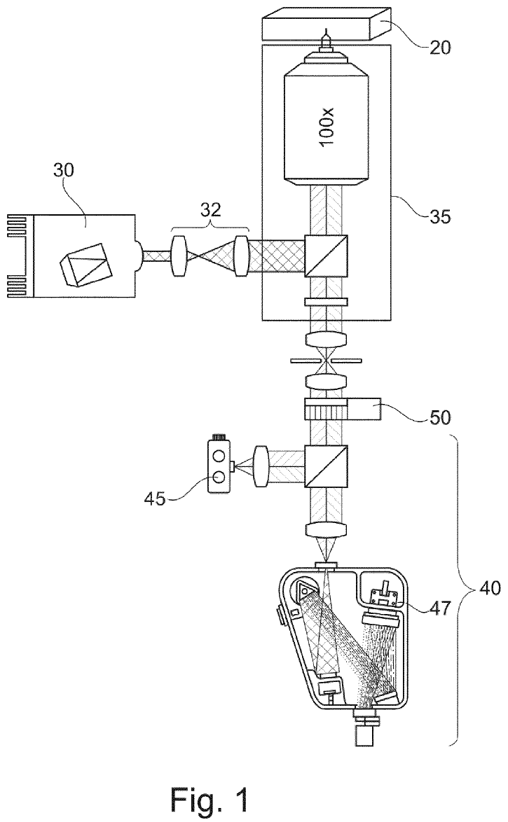

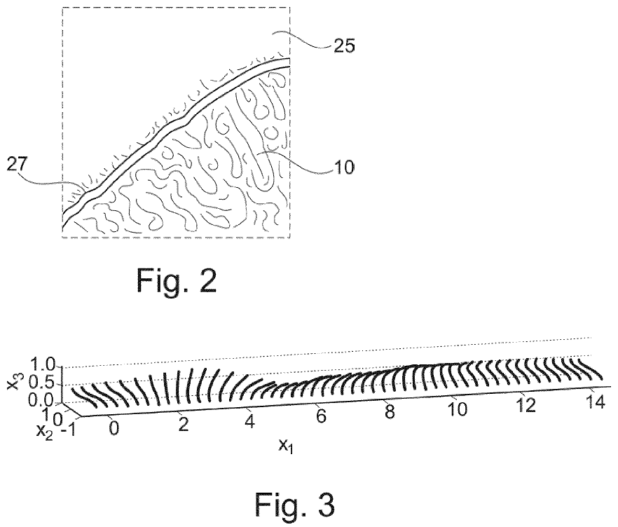

### DETAILED DESCRIPTION

An example of a process for studying a cluster of ciliated epithelial cells according to the invention is described below.

Firstly, ciliated epithelial cells 10 are cultured in a chamber 20 containing a physiological solution 25 containing nanorods. The ciliated epithelial cells have cilia 27 on their surface which, in the case of healthy cells, exhibit a permanent, periodic movement synchronized with that of neighboring cilia and those of neighboring cells, with a phase shift that increases with the distance. The movement of the cilia follows the motion of a metachronous wave, as represented in three dimensions in FIG. 3. The culture chamber may be a microscope plate, a Petri dish or, preferentially, a microfluidic chamber.

The nanorods are made of europium-doped lanthanum phosphate (LaPO₄:Eu) with a rhabdophane or monazite crystal phase, preferably monazite, and include polyethylene glycol silane (PEG-silane) linking molecules on their surface. The nanorods in the physiological solution have a length distribution characterized by a mean length of between 100 nm and 500 nm with a standard deviation of less than or equal to 200 nm, and a larger cross-sectional dimension distribution characterized by an average larger dimension of between 20 nm and 5 nm with a standard deviation of less than or equal to 10 nm.

A measurement zone of chamber 20 is then illuminated by a light source, notably a substantially monochromatic laser 30 at a wavelength of 395 nm, whose emitted light is transmitted in a conventional manner to the light input of an optical microscope 35 so as to illuminate the chamber in the measurement zone. The light source 30 excites the photoluminescence of the nanorods at a wavelength of 395 nm, corresponding to the excitation peak of the ⁷F₀-⁵L₆ transition for Eu³⁺. The spectra between 580 nm and 720 nm of the photoluminescence light emitted by the nanorods excited in the measurement zone are then measured at two different polarization angles, here orthogonal to each other, using a measuring system 40 and a rotating polarizer 50 arranged between the microfluidic chamber 20 and the measuring system 40. The light emitted by the nanorods is transmitted to the measuring system 40 via the optical microscope 35, with the polarizer 50 arranged between the optical microscope 35 and the measuring system 40. The optical microscope may include a scanning system, notably a piezoelectric scanning device.

The largest dimension of the measuring zone is less than or equal to 2 μm, and the resolution of the measuring system is less than or equal to 1 μm³.

Scanning of the microfluidic chamber in two directions using the scanning system allows measurement at any point in the measuring chamber.

The photoluminescence spectra emitted by the nanorods in each measurement zone between 580 and 720 nm at the two polarization angles then make it possible to determine a parameter of order f, of between 0 and 1, quantifying the local dispersion of the nanorod orientation, 0 corresponding to a disordered orientation of the nanorods and 1 to totally aligned nanorods, and a directrix \vec{n} corresponding to the average orientation of the nanorods as described on pages 6 and 7 of the appendix to the article Kim, J., Michelin, S., Hilbers, M. et al. Supplementary information to “Monitoring the orientation of rare-earth-doped nanorods for flow shear tomography”, Nature Nanotech. 12, 914-919 (2017).

The value of the shear rate in the measurement zone at each point in the measurement chamber is then determined from the parameter of order f, and the shear orientation is given by the average orientation of the nanorods \vec{n}, as described on pages 7 to 11 of the appendix to the article Kim, J., Michelin, S., Hilbers, M. et al. Supplementary information to “Monitoring the orientation of rare-earth-doped nanorods for flow shear tomography”, Nature Nanotech. 12, 914-919 (2017).

Shear mapping at the interface between the fluid medium and the ciliated cell(s) may then be established. Such a mapping is compared with the mapping of healthy ciliated cells, and it may then be determined whether the ciliated cells studied have functioning close to or remote from that of reference healthy cells. Specifically, in the physiological state, the cilia located at the apical part of the ciliated epithelial cells beat synchronously and generate a shear force on the fluid, as described in the article Bottier, M. et al. “A new index for characterizing micro-bead motion in a flow induced by ciliary beating: part II, modeling”, PLOS Comput. Biol. 13 (7): e1005552 (2017). Thus, when the cilia beat in an asynchronous manner or when beating is absent, the shear detected by the method described above is weak or even non-existent. The information determined by the method described thus makes it possible to characterize the beating efficiency of the ciliated cells studied.

As a variant, the photoluminescence spectra emitted by the nanorods in a measurement range between 580 and 720 nm at both polarization angles is measured continuously over time with acquisitions spaced at a time of about 1 μs. The shear rate value and shear orientation are then determined for each measurement as described previously to deduce the dynamic behavior of the cell sample 10, notably the movement of the cilia 27 thereof, in the measurement zone.

The invention is not limited to the example just described. For example, the biological material is not limited to ciliated airway epithelial cells, but may also be applied to the measurement of ciliated cells of the endosalpinx of the Fallopian tubes, to flagellated cells, such as sperm or embryos, or even to organisms such as paramecia (unicellular ciliated protozoa) used as a model for the study of cilia.

As a variant, it is also possible to determine a local orientation characteristic of the nanorods from the measurement of the birefringence of the nanorods in the fluid medium at the interface with the biological material, to deduce the local shear rate.

As a variant, the nanoparticles are anisotropically shaped nanoparticles other than the nanorods mentioned, the important point being that a physical signature of the orientation of the latter exists and is sufficiently strong and characteristic to allow identification of the general orientation of the nanoparticles and deduction of the shear rate.

As a variant, the nanoparticles include other linking molecules on the surface, allowing not only good dispersion of the nanoparticles in the fluid medium but also low toxicity for the biological material, or are free of linking molecules on the surface, in particular when the nanoparticles are in sufficiently stable suspension.

1. A process for studying the behavior of a biological material contained in a medium containing a plurality of anisotropically-shaped nanoparticles dispersed therein, the process involving:
(i) determining at least one orientation characteristic of the nanoparticles in a measurement zone at the interface between the fluid medium and the biological material, the orientation resulting at least partly from the interaction of the fluid medium and the biological material,
(ii) determining a mean shear characteristic of the fluid medium in the measurement zone from said at least one nanoparticle orientation characteristic determined in this measurement zone,
(iii) determining a characteristic of the cell sample from the mean shear rate thus determined in the measurement zone.

2. The process as claimed in claim 1, in which the cell sample is composed of at least one cell cluster.

3. The process as claimed in claim 1, in which the nanoparticles exhibit polarized photoluminescence emission.

4. The process as claimed in claim 1, in which the nanoparticles are rare-earth-doped nanorods.

5. The process as claimed in claim 1, in which the nanoparticles are rare-earth-doped.

6. The process as claimed in claim 1, in which the nanoparticles have a mean length of less than or equal to 1 μm, and a standard deviation of the nanoparticle length distribution of less than or equal to 100 nm.

7. The process as claimed in claim 1, in which the nanoparticles have an aspect ratio of greater than or equal to 3, better still greater than or equal to 10.

8. The process as claimed in claim 1, in which the nanoparticle concentration in the fluid medium is less than or equal to 10% as a volumetric fraction of the fluid medium.

9. The process as claimed in claim 1, which involves determining at least two characteristics of the nanoparticle orientation, the nanoparticle orientation and the associated order parameter characteristic of the dispersion of the nanoparticle orientation relative to the orientation in the measurement zone.

10. The process as claimed in claim 9, which involves determining the shear direction and shear value in the measurement zone from the two nanoparticle orientation characteristics determined in this measurement zone.

11. The process as claimed in claim 1, in which the nanoparticles are photoluminescent and the step for determining the orientation characteristic of the nanoparticles involves:
photoluminescence excitation of the nanoparticles by a light source, causing the nanoparticles to emit photoluminescent light, and
measuring at least two items of spectral information of the polarized photoluminescence light at one or more different polarizations in the measurement zone,
determining the orientation characteristic of the nanoparticles from the measurements of the at least two items of spectral information of the polarized photoluminescence light at one or more different polarizations.

12. The process as claimed in claim 11, in which the nanoparticles are europium-doped LaPO₄ nanorods, at least two measured items of spectral information corresponding to the spectra between 570 and 720 nm in which the transition bands of the Eu³⁺ ion are located.

13. The process as claimed in claim 11, which involves measuring the intensity of light polarized at one or more different polarization angles at least two different given wavelengths, the given wavelengths corresponding in the spectrum of light emitted by the nanoparticles to two different intensity peaks in the spectrum of the photoluminescence light.

14. The process as claimed in claim 1, which involves:
scanning the fluid medium in at least two directions by the measuring system,
determining at least one nanoparticle orientation characteristic at each position of the measuring system, and
mapping the shear rate in the fluid medium from said at least one nanoparticle orientation characteristic measured at each position of the measuring system.

15. The process as claimed in claim 1, which involves comparing the shear characteristic in the determined measurement zone or the mapping of the shear characteristic to a reference shear characteristic or a reference mapping of the pre-established shear characteristic.

16. A device for studying a cell sample including:
a fluidic chamber including:
a fluid medium, and
a plurality of nanoparticles dispersed in the fluid medium,
a system for measuring at least one item of information characteristic of the orientation of the nanoparticles in a measurement zone at the interface between the fluid medium and the cell sample.

17. The device as claimed in claim 16, which includes or is connected to a processor configured to determine the shear characteristic in the measurement zone from information characteristic of the nanoparticle orientation determined by the measurement system in this measurement zone.

18. The process as claimed in claim 1, in which the nanoparticles are europium-doped, oxide or fluoride, lanthanum phosphate (LaPO₄), sodium yttrium fluoride (NaYF₄) or derivatives thereof.

19. The process as claimed in claim 1, in which the nanoparticles are europium-doped LaPO₄ in rhabdophane crystalline phase or in monazite
